(19) United States
(12) Patent Application Publication
(10) Pub. No.: US 2007/0160640 A1
(43) Pub. Date: Jul. 12, 2007

(54) Halofuginone delivering vascular medical devices

(76) Inventors: Eun-Hyun Jang, Cambridge, MA (US); Young-Ho Song, Natick, MA (US); Robert A. Herrmann, Boston, MA (US); Anurag Singhal, Natick, MA (US); Brad Pires, Dartmouth, MA (US); Andrew Saati, Framingham, MA (US)

Correspondence Address: MAYER & WILLIAMS PC, 251 NORTH AVENUE WEST, 2ND FLOOR, WESTFIELD, NJ, 07090, US

(21) Appl. No.: 11/330,448

(22) Filed: Jan. 12, 2006

(51) Int. Cl. A61F 2/02

(52) U.S. Cl. 424/423

(57) ABSTRACT

A medical device comprising halofuginone is provided. The medical device is adapted for implantation or insertion into a blood vessel and it provides a cumulative, in vivo, 14 day release that lies between 0.02 μg and 0.2 μg of halofuginone per mm² of stent surface area.

### FIELD OF THE INVENTION

The present invention relates generally to medical devices, and more particularly to implantable or insertable medical devices which release halofuginone.

### BACKGROUND OF THE INVENTION

The in vivo delivery of a biologically active agent within the body of a patient is common in the practice of modern medicine. In vivo delivery of biologically active agents is often implemented using medical devices that may be temporarily or permanently placed at a target site within the body. These medical devices can be maintained, as required, at their target sites for short or prolonged periods of time, delivering biologically active agents at the target site.

For example, drug delivery from stents for the treatment of restenosis is widely accepted. Commercially available drug eluting coronary stents include those available from Boston Scientific Corp. (TAXUS), Johnson & Johnson (CYPHER), and others.

Unfortunately, only a few products have been successful to date, in part, due to the inability to create products with safe dose and release kinetics. For coronary stents with polymeric drug-eluting coatings, dose and release kinetics are affected, for example, by the physiochemical properties of the drug and the polymeric carrier, by the interactions between the drug and carrier, and by the geometry of the system.

Halofuginone is a novel inhibitor of collagen synthesis. It is effective in preventing extracellular matrix formation and cell proliferation. Traditionally, it has been used in animal feeds as an antibiotic agent. It has also been approved for the treatment of scleroderma, and is expected to gain approval for use in other areas, for example, the treatment of restenosis, cancer, fibroproliferative diseases and/or other diseases and conditions. In each of case, however, there will be a need to determine what dosages are safe.

### SUMMARY OF THE INVENTION

According to an aspect of the present invention, a medical device is provided, which contains halofuginone. The medical device is adapted for implantation or insertion into a blood vessel, and it provides a cumulative, 14 day, in vivo release that lies between 0.02 μg and 0.2 μg of halofuginone per mm² of stent surface area.

An advantage of the present invention is that halofuginone dosage ranges have been determined, which are safe for vascular administration in mammals.

Another advantage of the present invention is that medical devices have been created, which provide such dosage ranges.

These and other aspects, embodiments and advantages of the present invention will become immediately apparent to those of ordinary skill in the art upon review of the Detailed Description and Claims to follow.

### BRIEF DESCRIPTION OF THE DRAWINGS

FIG. 1 is a graph of cumulative release of halofuginone as a function of time for two coating compositions.

FIGS. 2-4 are photographs illustrating the histology for an uncoated stent, a low-dose stent and a high-dose stent, respectively.

### DETAILED DESCRIPTION OF THE INVENTION

According to an aspect of the invention, medical devices are provided which are adapted for implantation or insertion into a blood vessel of a subject and which contact an inner surface of the blood vessel wall upon implantation or insertion. Typical subjects (or “patients”) are vertebrate subjects, more typically mammalian subjects, and even more typically human subjects.

The medical device has a release profile that provides a cumulative in vivo release, at 14 days, which lies between 0.02 and 0.2 μg (e.g., from 0.02 μg to 0.04 μg to 0.1 μg to 0.2 μg) of halofuginone, per mm² of stent surface area.

“Cumulative in vivo release after a given time period”, as defined herein, is either the cumulative release that occurs after being implanted in vivo for the time period selected or is the cumulative release that is measured upon placing the device in a surrogate environment, specifically the cumulative release that is measured upon immersing the medical device for the time period selected (e.g., 14 days) in a 37° C. solution of phosphate buffered saline (PBS) having a of pH 7.4, to which has been added 0.5 g Tween® 20 (known generically as Polyoxyethylene(20)sorbitan monolaurate) per liter. An example of one such test is given below in the Example.

The cumulative release need not be linear, and in various embodiments, the majority of the cumulative amount of drug released during the first 14 days of immersion has already occurred by the 7^{th} day of immersion or even earlier. For example, the device's release profile may result in a cumulative in vitro release of drug that is measured during the first 3 days, which is 90% or more of the cumulative in vitro release of drug that is measured during the first 14 days. As another example, the device's release profile may result in a cumulative in vitro release of drug that is measured during the first 2 days that is 90% or more of the cumulative in vitro release of drug that is measured during the first 14 days. As another example, the device's release profile may result in a cumulative in vitro release of drug that is measured during the first 24 hours that is 75% or more of the cumulative in vitro release of drug that is measured during the first 14 days. As yet another example, the device's release profile may result in a cumulative in vitro release of drug that is measured during the first 12 hours that is 75% or more of the cumulative in vitro release of drug that is measured during the first 14 days.

As used herein, the term “halofuginone” includes halofuginone in free base form, halofuginone in salt form (e.g., halofuginone hydrobromide, halofuginone lactate, etc.), and mixtures thereof.

Vascular medical devices benefiting from the present invention include vascular stents such as coronary artery stents, and peripheral vascular stents such as cerebral stents.

In some embodiments, medical devices in accordance with the present invention contain release regions. Release regions are material regions (e.g., layers, etc.), which control the release of halofuginone disposed beneath or within the same (and, optionally, one or more additional therapeutic agents as well). Release regions in accordance with the present invention include carrier regions and barrier regions. By “carrier region” is meant a release region that contains a therapeutic agent (e.g., halofuginone) and from which the therapeutic agent is released. For example, in some embodiments, the carrier region may constitute the entirety of the medical device (e.g., provided in the form of a polymeric stent body that is loaded with therapeutic agent). In other embodiments, the carrier region corresponds to only a portion of the device (e.g., a polymeric carrier layer overlying a medical device substrate such as a stent body). By “barrier region” is meant a region which is disposed between a source of therapeutic agent and a site of intended release, and which controls the rate at which therapeutic agent is released. For example, in some embodiments, the medical device consists of a barrier region that surrounds a source of halofuginone. In other embodiments, the barrier region (e.g., a polymeric layer) is disposed over a source of halofuginone, which is in turn disposed over all or a portion of a medical device substrate.

Barrier layers and carrier layers can be provided over underlying substrates at a variety of locations, and in a variety of shapes (e.g., in desired patterns, for instance, using appropriate masking techniques, such as lithographic techniques), and they can be formed from a variety of materials, including various polymeric materials as discussed further below. As used herein a “layer” of a given material is a region of that material whose thickness is small compared to both its length and width. As used herein a layer need not be planar, for example, taking on the contours of an underlying substrate. Layers can be discontinuous (e.g., patterned). Terms such as “film,” “layer” and “coating” may be used interchangeably herein.

Materials for use as underlying medical device substrates include (a) organic materials (e.g., materials containing 50 wt % or more organic species) such as polymeric materials and (b) inorganic materials (e.g., materials containing 50 wt % or more inorganic species), such as metallic materials (e.g., metals and metal alloys) and non-metallic materials (e.g., including carbon, semiconductors, glasses and ceramics, which may contain various metal- and non-metal-oxides, various metal- and non-metal-nitrides, various metal- and non-metal-carbides, various metal- and non-metal-borides, various metal- and non-metal-phosphates, and various metal- and non-metal-sulfides, among others).

Specific examples of non-metallic inorganic materials may be selected, for example, from materials containing one or more of the following: metal oxides, including aluminum oxides and transition metal oxides (e.g., oxides of titanium, zirconium, hafnium, tantalum, molybdenum, tungsten, rhenium, and iridium); silicon; silicon-based ceramics, such as those containing silicon nitrides, silicon carbides and silicon oxides (sometimes referred to as glass ceramics); calcium phosphate ceramics (e.g., hydroxyapatite); carbon; and carbon-based, ceramic-like materials such as carbon nitrides.

Specific examples of metallic inorganic materials may be selected, for example, from metals (e.g., biostable metals such as gold, platinum, palladium, iridium, osmium, rhodium, titanium, tantalum, tungsten, and ruthenium, and bioresorbable metals such as magnesium), metal alloys comprising iron and chromium (e.g., stainless steels, including platinum-enriched radiopaque stainless steel), alloys comprising nickel and titanium (e.g., Nitinol), alloys comprising cobalt and chromium, including alloys that comprise cobalt, chromium and iron (e.g., elgiloy alloys), alloys comprising nickel, cobalt and chromium (e.g., MP 35N) and alloys comprising cobalt, chromium, tungsten and nickel (e.g., L605), and alloys comprising nickel and chromium (e.g., inconel alloys).

Specific examples of organic materials may be selected, for example, from the polymeric material listed below for use in forming polymeric release regions.

Where implantable or insertable medical devices are provided which contain polymeric release regions that regulate the release of halofuginone, the release profile associated with such devices may be modified, for example, by changing the chemical composition, size, number and/or position of the polymeric release regions within the device, among other parameters. For example, where a polymeric carrier layer is used, the release profile of polymeric carrier layer may be affected by the concentration of therapeutic agent (e.g., halofuginone) within the carrier layer, by the polymer composition of the carrier layer, by the volume of the carrier layer, by the surface area of the carrier layer, by the presence of any barrier layers between the carrier layer and the site of release, and so forth. In this regard, multiple carrier or barrier layers of the invention, either having the same or different content (e.g., different polymer and/or halofuginone content), may be stacked on top of one another, may be positioned laterally to one another, and so forth.

For tubular devices such as stents (which can comprise, for example, a laser or mechanically cut tube, one or more braided, woven, or knitted filaments, etc.), polymeric release layers can be provided on the abluminal surfaces, on the luminal surfaces and/or on the lateral surfaces between the luminal and abluminal surfaces (including the ends). Moreover, release layers can control the release of the same or differing underlying biologically active agent. It is therefore possible, for example, to release the same or different therapeutic agents at different rates from different locations on the medical device. As another specific example, it is possible to provide a tubular medical device (e.g., a vascular stent) having a release layer which contains or is disposed over an antithrombotic agent at its inner, luminal surface and a second release layer which contains or is disposed over halofuginone at its outer, abluminal surface (as well as on the ends, if desired).

By a “polymeric” region is meant a region that contains polymers, commonly at least 50 wt % to 75 wt % to 90 wt % to 95 wt % to 97.5 wt % to 99 wt %, or even more, polymers. “Polymers” are molecules that contain multiple copies of the same or differing constitutional units, commonly referred to as monomers, and typically containing from 5 to 10 to 25 to 50 to 100 or more constitutional units. Depending on the number and nature of the polymer chains making them up, the polymers for use in the present invention may have a variety of architectures, including cyclic, linear and branched architectures. Branched architectures include star-shaped architectures (e.g., architectures in which three or more chains emanate from a single branch point), comb architectures (e.g., architectures having a main chain and a plurality of side chains) and dendritic architectures (e.g., arborescent and hyperbranched polymers), among others. The polymers may contain, for example, homopolymer chains, which contain multiple copies of a single constitutional unit, and/or copolymer chains, which contain multiple copies of at least two dissimilar constitutional units, which units may be present in any of a variety of distributions including random, statistical, gradient, and periodic (e.g., alternating) distributions. “Block copolymers” are polymers containing two or more differing polymer chains, for example, selected from homopolymer chains and random and periodic copolymer chains.

Specific polymers for use in forming polymeric release regions may be selected, for example, from the following: polycarboxylic acid polymers and copolymers including polyacrylic acids; acetal polymers and copolymers; acrylate and methacrylate polymers and copolymers (e.g., n-butyl methacrylate); cellulosic polymers and copolymers, including cellulose acetates, cellulose nitrates, cellulose propionates, cellulose acetate butyrates, cellophanes, rayons, rayon triacetates, and cellulose ethers such as carboxymethyl celluloses and hydroxyalkyl celluloses; polyoxymethylene polymers and copolymers; polyimide polymers and copolymers such as polyether block imides and polyether block amides, polyamidimides, polyesterimides, and polyetherimides; polysulfone polymers and copolymers including polyarylsulfones and polyethersulfones; polyamide polymers and copolymers including nylon 6,6, nylon 12, polycaprolactams and polyacrylamides; resins including alkyd resins, phenolic resins, urea resins, melamine resins, epoxy resins, allyl resins and epoxide resins; polycarbonates; polyacrylonitriles; polyvinylpyrrolidones (cross-linked and otherwise); polymers and copolymers of vinyl monomers including polyvinyl alcohols, polyvinyl halides such as polyvinyl chlorides, ethylene-vinyl acetate copolymers (EVA), polyvinylidene chlorides, polyvinyl ethers such as polyvinyl methyl ethers, polystyrenes, styrene-maleic anhydride copolymers, vinyl-aromatic-alkylene copolymers, including styrene-butadiene copolymers, styrene-ethylene-butylene copolymers (e.g., a polystyrene-polyethylene/butylene-polystyrene (SEBS) copolymer, available as Kratong G series polymers), styrene-isoprene copolymers (e.g., polystyrene-polyisoprene-polystyrene), acrylonitrile-styrene copolymers, acrylonitrile-butadiene-styrene copolymers, styrene-butadiene copolymers and styrene-isobutylene copolymers (e.g., polyisobutylene-polystyrene and polystyrene-polyisobutylene-polystyrene block copolymers such as those disclosed in U.S. Pat. No. 6,545,097 to Pinchuk), polyvinyl ketones, polyvinylcarbazoles, and polyvinyl esters such as polyvinyl acetates; polybenzimidazoles; ethylene-methacrylic acid copolymers and ethylene-acrylic acid copolymers, where some of the acid groups can be neutralized with either zinc or sodium ions (commonly known as ionomers); polyalkyl oxide polymers and copolymers including polyethylene oxides (PEO); polyesters including polyethylene terephthalates and aliphatic polyesters such as polymers and copolymers of lactide (which includes lactic acid as well as d-,1- and meso lactide), epsilon-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, and 6,6-dimethyl-1,4-dioxan-2-one (a copolymer of poly(lactic acid) and poly(caprolactone) is one specific example); polyether polymers and copolymers including polyarylethers such as polyphenylene ethers, polyether ketones, polyether ether ketones; polyphenylene sulfides; polyisocyanates; polyolefin polymers and copolymers, including polyalkylenes such as polypropylenes, polyethylenes (low and high density, low and high molecular weight), polybutylenes (such as polybut-1-ene and polyisobutylene), polyolefin elastomers (e.g., santoprene), ethylene propylene diene monomer (EPDM) rubbers, poly-4-methyl-pen-1-enes, ethylene-alpha-olefin copolymers, ethylene-methyl methacrylate copolymers and ethylene-vinyl acetate copolymers; fluorinated polymers and copolymers, including polytetrafluoroethylenes (PTFE), poly(tetrafluoroethylene-co-hexafluoropropene) (FEP), modified ethylene-tetrafluoroethylene copolymers (ETFE), and polyvinylidene fluorides (PVDF); silicone polymers and copolymers; thermoplastic polyurethanes (TPU); elastomers such as elastomeric polyurethanes and polyurethane copolymers (including block and random copolymers that are polyether based, polyester based, polycarbonate based, aliphatic based, aromatic based and mixtures thereof, examples of commercially available polyurethane copolymers include Bionate®, Carbothane®, Tecoflex®, Tecothane®, Tecophilic®, Tecoplast®, Pellethane®, Chronothane® and Chronoflex®); p-xylylene polymers; polyiminocarbonates; copoly(ether-esters) such as polyethylene oxide-polylactic acid copolymers; polyphosphazines; polyalkylene oxalates; polyoxaamides and polyoxaesters (including those containing amines and/or amido groups); polyorthoesters; biopolymers, such as polypeptides, proteins, polysaccharides and fatty acids (and esters thereof), including fibrin, fibrinogen, collagen, elastin, chitosan, gelatin, starch, glycosaminoglycans such as hyaluronic acid; as well as further copolymers and blends of the above.

In certain advantageous embodiments, block copolymers are used in the polymeric release regions of the present invention that contain (a) one or more low T_{g} (glass transition temperature) polymer blocks and (b) one or more high T_{g} polymer blocks. Block copolymers having low and high T_{g} polymer blocks are known to possess many interesting physical properties due to the presence of a low T_{g} phase, which is soft and elastomeric at room (and body) temperature, and a high T_{g} phase, which is hard at these temperatures. As used herein, “low T_{g} polymer blocks” are those that display a T_{g} that is below ambient temperature, more typically 20° C. to 0° C. to −25° C. to −50° C. or below. Conversely, elevated or “high T_{g} polymer blocks” are those that display a glass transition temperature that is above ambient temperature, more typically 50° C. to 75° C. to 100° C. or above. “Ambient temperature” is typically 25° C.-45° C., and includes body temperature (e.g., 35° C.-40° C.). T_{g} can be measured by any of a number of techniques including differential scanning calorimetry (DSC), dynamic mechanical analysis (DMA), or dielectric analysis (DEA).

Block copolymer configurations may vary widely and include, for example, the following configurations (in which high T_{g} polymer chains, H, and low T_{g} polymer chains, L, are used for illustrative purposes, although other blocks having different characteristics can clearly be substituted): (a) block copolymers having alternating chains of the type (HL)ₘ, L(HL)ₘ and H(LH)ₘ where m is a positive whole number of 1 or more, (b) multiarm copolymers such as X(LH)ₙ, and X(HL)ₙ, where n is a positive whole number of 2 or more, and X is a hub species (e.g., an initiator molecule residue, a residue of a molecule to which preformed polymer chains are attached, etc.), and (c) comb copolymers having a L chain backbone and multiple H side chains and vice versa (i.e., having an H chain backbone and multiple L side chains).

Some specific examples of low T_{g} blocks include low T_{g} polyalkylene blocks, for example, those comprising ethylene, propylene, butylene, isobutylene, isoprene and/or butadiene monomers, polysiloxane blocks, low T_{g} poly(halogenated alkylene) blocks, low T_{g} polyacrylate blocks, low T_{g} polymethacrylate blocks, low T_{g} poly(vinyl ether) blocks, low T_{g} poly(cyclic ether) blocks, among others. Some specific examples of high T_{g} blocks include vinyl aromatic blocks, such as those made from styrenic monomers (e.g., styrene and/or styrene derivatives such as α-methylstyrene, ring-alkylated styrenes, ring-halogenated styrenes or other substituted styrenes where one or more substituents are present on the aromatic ring), high T_{g} polyacrylate blocks, high T_{g} polymethacrylate blocks, poly(vinyl alcohol) blocks, high T_{g} poly(vinyl ester) blocks, high T_{g} poly(vinyl amine) blocks, high T_{g} poly(vinyl halide) blocks and high T_{g} poly(alkyl vinyl ethers), among others. Further specific examples of low and high T_{g} blocks may be found, for example, in U.S. Patent Application No. 2005/0064011, which is hereby incorporated by reference in its entirety.

Specific examples of beneficial block copolymers include those having polyalkylene blocks and poly(vinyl aromatic) blocks, such as block copolymers containing polyisobutylene and polystyrene blocks, for example, polystyrene-polyisobutylene-polystyrene triblock copolymers (SIBS copolymers), described in U.S. Pat. No. 6,545,097 to Pinchuk et al., which is hereby incorporated by reference in its entirety. These copolymers have proven to be valuable elastomers for use implantable or insertable medical device applications due to their excellent strength, biocompatibility and biostability. For example, these copolymers exhibit high tensile strength, which frequently ranges from 2,000 to 4,000 psi or more. Biocompatibility, including vascular compatibility, of these materials has been demonstrated by their tendency to provoke minimal adverse tissue reactions (e.g., as measured by reduced macrophage activity). In addition, these polymers are generally hemocompatible as demonstrated by their ability to minimize thrombotic occlusion of small vessels when applied as a coating on coronary stents. SIBS copolymers are also biostable, resisting cracking and other forms of degradation throughout the body, including the gastrointestinal tract. Other specific examples of block copolymers of polyisobutylene and polystyrene include arborescent polyisobutylene-polystyrene block copolymers such as those described in Kwon et al., “Arborescent Polyisobutylene-Polystyrene Block Copolymers-a New Class of Thermoplastic Elastomers,” Polymer Preprints, 2002, 43(1), 266, the disclosure of which is incorporated by reference

Other examples of block copolymers having poly(vinyl aromatic) blocks and polyalkylene blocks include polystyrene-poly(ethylene/butylene)-polystyrene (SEBS) block copolymer, available as Kraton™ G series polymers from Kraton Polymers.

As noted above, the medical devices of the present invention contain halofuginone and may optionally contain one or more additional therapeutic agents as well. “Therapeutic agents,” “drugs,” “pharmaceutically active agents,” “pharmaceutically active materials,” and other related terms may be used interchangeably herein. These terms include genetic therapeutic agents, non-genetic therapeutic agents and cells.

Optional therapeutic agents useful for the practice of the present invention may be selected, for example, from those described in paragraphs [0040] to [0046] of commonly assigned U.S. Patent Application Pub. No. 2003/0236514, the disclosure of which is hereby incorporated by reference.

Various techniques are available for forming medical devices in accordance with the present invention. For instance, in embodiments where a polymeric release region is employed to control the release of the halofuginone (and any additional therapeutic agents), techniques include solvent based techniques and thermoplastic techniques.

For example, in embodiments where the polymer(s) making up the release region have thermoplastic characteristics, a variety of standard thermoplastic processing techniques can be used to form polymeric regions of various shapes, including compression molding, injection molding, blow molding, spinning, vacuum forming and calendaring, as well as extrusion into sheets, fibers, rods, tubes and other cross-sectional profiles of various lengths. Using these and other thermoplastic processing techniques, entire articles or portions thereof can be made.

In other embodiments, solvent-based techniques are used to form polymeric regions of various shapes. Using these techniques, a region can be formed by providing a solution that contains a solvent and polymer(s) of choice. The solvent that is ultimately selected will contain one or more solvent species, which are generally selected based on their ability to dissolve the polymers making up the polymeric region, as well as other factors, including drying rate, surface tension, etc. Generally, several solvents will be tested to see which provides regions having the best characteristics.

Preferred solvent-based techniques include, but are not limited to, solvent casting techniques, spin coating techniques, web coating techniques, solvent spraying techniques, dipping techniques, techniques involving coating via mechanical suspension including air suspension, ink jet techniques, electrostatic techniques, and combinations of these processes.

In various embodiments of the invention, a solution (where solvent-based processing is employed) or melt (where thermoplastic processing is employed) is applied to a substrate to form a desired region. For example, the substrate can correspond to all or a portion of a medical article surface to which a layer is applied. The substrate can also be, for example, a template, such as a mold, from which the region is removed after solidification. In other embodiments, for example, extrusion and co-extrusion techniques, one or more regions may be formed without the aid of a substrate.

So long as the halofuginone and/or any other optional agents are stable under processing conditions, then they may be provided within the solution or melt and co-processed along with the other compounds to form carrier regions. Alternatively, halofuginone and/or other optional agents may also be introduced subsequent to the formation of the polymeric region in some instances. For instance, in some embodiments, the halofuginone and/or any optional agents are dissolved or dispersed within a solvent, and the resulting solution contacted with a previously formed polymeric region (e.g., using one or more of the application techniques described above, such as dipping, spraying, etc.).

Polymeric regions are provided over therapeutic-agent-containing regions in some embodiments of the invention (e.g., where the polymeric region acts as a barrier region, to slow the release of the halofuginone). In these embodiments, for example, a polymeric region can be formed over a therapeutic-agent-containing region, for instance, using one of the solvent based or thermoplastic techniques described above. Alternatively, a previously formed polymeric region may be adhered over a halofuginone containing region.

### EXAMPLE

A coating is made using halofuginone (Hfg) in free base form, obtained from Collgard Biopharmaceutical with a particle size of approximately 2 microns in diameter. Polystyrene-polyisobutylene-polystyrene triblock copolymer (SIBS) is prepared as described in United States Patent Application 20020107330 and U.S. Pat. No. 6,545,097 entitled “Drug delivery compositions and medical devices containing block copolymer”. Phosphate buffered saline (PBS) with Tween® 20, pH 7.4, is obtained from Sigma-Aldrich.

A first coating solution is made by suspending 0.3 wt % Hfg particles in a dichloromethane/toluene (94 wt %/5 wt %, respectively) solution containing 0.7 wt % SIBS. A second coating solution is made by suspending 0.1 wt % Hfg in a dichloromethane/toluene (94 wt %/5 wt %, respectively) solution containing 0.9 wt % SIBS. The coating solution was sprayed onto the inner and outer surfaces of a bare stainless steel stent and dried in a vacuum oven at 40° C. for 1 hour. All stents are 8 mm Express™ stents, available from Boston Scientific, Inc., Natick, Mass., USA. Different doses may be achieved by varying the SIBS/Hfg ratios as above and by the total amount of coating on a single stent. For example, a high-dose stent is formed by using the first solution described above to coat a bare stent to a coating weight of 600 μg (180 μg Hfg, 420 μg SIBS), whereas a low-dose stent is formed by coating a bare stent with the second solution to a coating weight of 600 μg (60 μg Hfg, 540 μg SIBS).

In vitro release was determined by placing coated stents into PBS/Tween® 20 solution at pH 7.4 at 37° C. Aliquots of the release medium were tested at different time points up to 2 weeks for the Hfg with quantification using HPLC. Results are presented in FIG. 1. With each of the above high- and low-dose formulations, less than 10% of the total drug on the stent is released over a period of 24 hours.

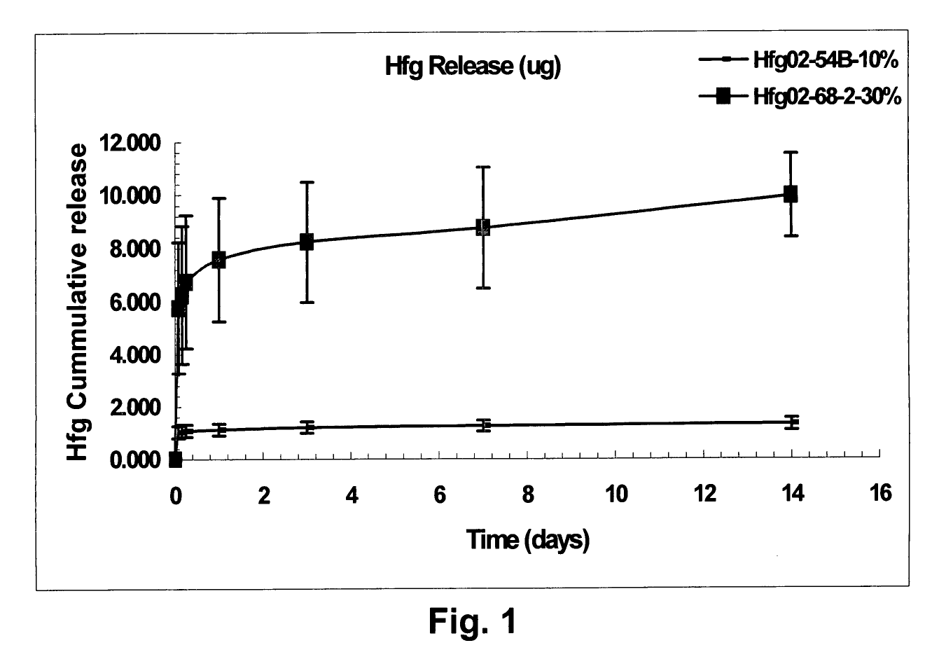

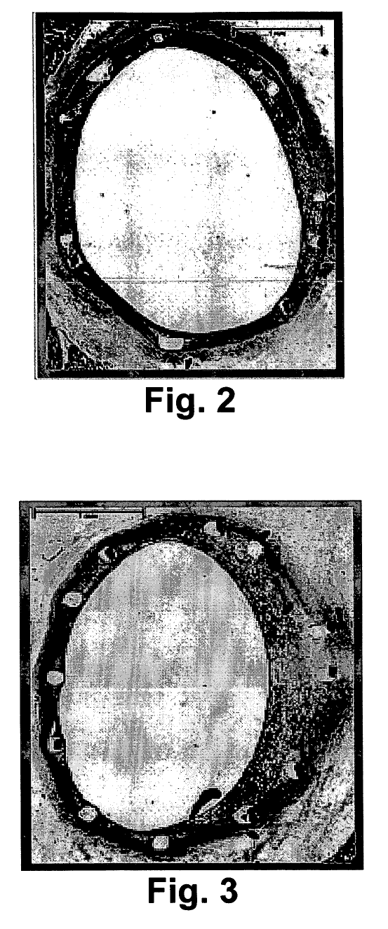

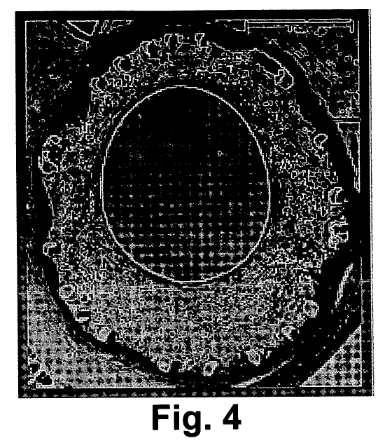

Stents having the high-and low-dose coatings described above, as well as an uncoated control stent, are implanted in porcine coronary arteries for 28 days, explanted, and the stented vessels are examined histologically. Photographs of the stented porcine coronary vessels (Trichrome staining) are presented in FIG. 2 (uncoated stent), FIG. 3(low-dose stent) and FIG. 4 (high-dose stent).

Data from these studies demonstrate that at a high-dose of 12 μg releasing over 2 weeks in vitro (0.24 μg/mm²), there is a toxic response of the vessel wall. With similar release kinetics but a lower dose of 2 μg releasing over 2 weeks in vitro (2 μg/50 mm² stent surface area=0.04 μg/mm²), there was no significant response (i.e., there was no statistical difference in percent stenosis between the low-dose coated stent and the uncoated stent).

Although various embodiments are specifically illustrated and described herein, it will be appreciated that modifications and variations of the present invention are covered by the above teachings and are within the purview of the appended claims without departing from the spirit and intended scope of the invention.

1. A medical device comprising halofuginone, said medical device being adapted for implantation or insertion into a blood vessel and said medical device providing a cumulative, in vivo, 14 day release that lies between 0.02 μg and 0.2 μg of halofuginone per mm² of stent surface area.

2. The medical device of claim 1, wherein said medical device comprises a polymeric release region.

3. The medical device of claim 1, wherein said medical device comprises a polymeric carrier layer disposed over a medical device substrate, said polymeric carrier layer comprising a polymer and said halofuginone.

4. The medical device of claim 3, wherein said polymeric carrier layer comprises a plurality of different types of polymers and said halofuginone.

5. The medical device of claim 3, wherein said polymeric carrier layer comprises a copolymer and said halofuginone.

6. The medical device of claim 5, wherein said copolymer comprises an alkylene monomer and a styrenic monomer.

7. The medical device of claim 5, wherein said copolymer comprises isobutylene and styrene monomers.

8. The medical device of claim 5, wherein said copolymer is a block copolymer.

9. The medical device of claim 8, wherein said block copolymer comprises a high glass transition temperature block and a low glass transition temperature block.

10. The medical device of claim 8, wherein said block copolymer comprises a polystyrenic block and a polyalkylene block.

11. The medical device of claim 8, wherein said block copolymer comprises a polystyrene block and a polyisobutylene block.

12. The medical device of claim 8, wherein said block copolymer comprises a polystyrene-polyisobutylene-polystyrene triblock.

13. The medical device of claim 1, wherein said halofuginone comprises halofuginone in free base form.

14. The medical device of claim 1, wherein said halofuginone comprises halofuginone in salt form.

15. The medical device of claim 1, wherein said medical device is a stent.

16. The medical device of claim 15, wherein said stent is a metallic stent.

17. The medical device of claim 1, wherein said medical device comprises a polymeric carrier layer disposed over a stent, said polymeric carrier layer comprising a polymer and said halofuginone.

18. The medical device of claim 17, wherein said stent is a metallic stent, and wherein said polymeric carrier layer comprises said halofuginone and a block copolymer that comprises polystyrene and polyisobutylene blocks.

19. The medical device of claim 1, wherein said release profile provides a cumulative, in vivo, 3 day release that is 90% or more of said cumulative, in vivo, 14 day release.

20. The medical device of claim 1, wherein said release profile provides a cumulative, in vivo, 2 day release that is 90% or more of said cumulative, in vivo, 14 day release.

21. The medical device of claim 1, wherein said release profile provides a cumulative, vivo, 24 hour release that is 75% or more of said cumulative, in vivo, 14 day release.

22. The medical device of claim 1, wherein said release profile provides a cumulative, vivo, 12 hour release in vitro release after a period of 12 hours that is 75% or more of said cumulative, in vivo, 14 day release.
